(19) United States
(12) Patent Application Publication
(10) Pub. No.: US 2025/0188012 A1
(43) Pub. Date: Jun. 12, 2025

(54) PROCESS FOR THE STEREOSELECTIVE HYDROGENATION OF PHTHALATES

(71) Applicant: Evonik Oxeno GmbH & Co. KG, Marl (DE)

(72) Inventors: Ruiyang Qu, Changchun (CN); Soumyashree Jena, Sundargarh (IN); Ralf Jackstell, Rostock (DE); Kathrin Junge, Rostock (DE); Matthias Beller, Ostseebad Nienhagen (DE); Robert Franke, Marl (DE)

(73) Assignee: Evonik Oxeno GmbH & Co. KG, Marl (DE)

(21) Appl. No.: 18/969,198

(22) Filed: Dec. 4, 2024

(30) Foreign Application Priority Data
Dec. 8, 2023 (EP) 23215244.7

(51) Int. Cl. C07C 67/303; C07C 69/80

(52) U.S. Cl. CPC C07C 67/303; C07C 69/80

(57) ABSTRACT

The invention relates to a process for the stereoselective hydrogenation of phthalates, to a hydrogenation catalyst and to a hydrogenated phthalate.

The present invention relates to a process for the stereoselective hydrogenation of phthalates, to a hydrogenation catalyst and to a hydrogenated phthalate.

The present invention was based on the object of providing a process for the stereoselective hydrogenation of phthalates.

This object is achieved by a process according to claim 1.

Process comprising the process steps of:
a) initially charging a phthalate of formula (I):

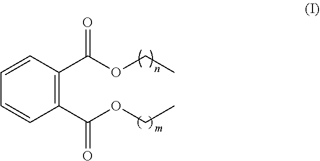

where n and m, each independently, are an integer from 0 to 12;
b) adding Pt/TiO₂;
c) feeding in H₂;
d) reacting the reaction mixture from a) to c), with conversion of the phthalate to a compound of formula (II):

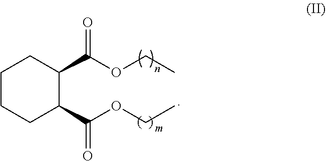

In one variant of the process, n and m are the same number.

In one variant of the process, n and m are a number from 4 to 8.

In one variant of the process, the phthalate has the structure (1):

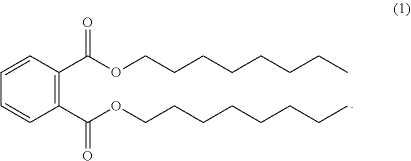 [SMILES: CCCCCCCCOC(=O)c1ccccc1C(=O)OCCCCCCCC]

In one variant of the process, Pt/TiO₂ is added in a range from 0.1 mol % of Pt to 5 mol % of Pt (based on the phthalate).

In one variant of the process, H₂ is fed in with a pressure in the range from 0.5 MPa (5 bar) to 10 MPa (100 bar).

In one variant of the process, the reaction of the reaction mixture is effected at a temperature in the range from 20° C. to 120° C.

In one variant of the process, the reaction of the reaction mixture is effected at a temperature in the range from 20° C. to 100° C.

In one variant of the process, the process comprises the additional process step c′):

c′) adding a solvent.

In one variant of the process, the solvent is cyclohexane.

As well as the process, the product obtained is also claimed.

Compound having the structure (1):

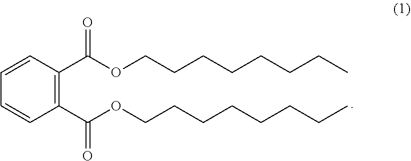 [SMILES: CCCCCCCCOC(=O)c1ccccc1C(=O)OCCCCCCCC]

Furthermore, the use of the catalyst used in a corresponding reaction is also claimed.

Use of a Pt/TiO₂ catalyst for catalysis of a stereoselective hydrogenation of an aromatic compound.

In one embodiment, the aromatic compound is a phthalate.

The invention is to be elucidated in more detail hereinafter with reference to a working example.

### EXPERIMENTAL DESCRIPTION

### Preparation of the Catalyst

A supported Pt catalyst with a Pt loading of 6% by weight was synthesized by the wet impregnation method. For a typical catalyst preparation (0.5 g), the desired amount of aqueous hydrogen hexachloroplatinic acid solution (7.5 mg Pt ml-1, Alfa Aesar) was diluted to 16 ml with deionized water and heated to 60° C. for 15 minutes in an oil bath with vigorous stirring (800 rpm), followed by the addition of TiO₂ support (P25, Sigma-Aldrich). The temperature was kept at 60° C. for 16 hours. After cooling, the water was removed by rotary evaporation. The material formed was then placed into a furnace at 110° C. overnight. The sample was thoroughly ground and then calcined for 4 hours at 450° C. with a heating rate of 10° C. min⁻¹. Finally, the sample was reduced in a through-flow tube furnace under constant flow of 5% H₂/Ar at 250° C. for 1 h with a heating rate of 5° C. min⁻¹.

### Stereoselective Hydrogenation of Di-n-Octyl Phthalate

a) Reaction with 0.5 mol % of Pt/TiO₂

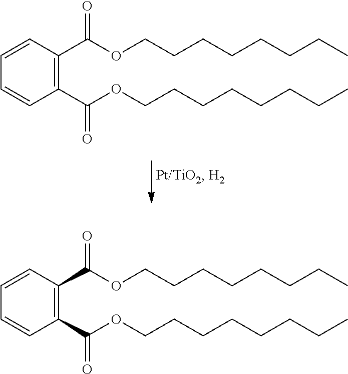

0.5 mol % of Pt/TiO₂ (6% by weight), substrate (195.3 mg, 0.5 mmol) and cyclohexane (2 ml) were added to a 4 ml screw-top reaction vessel with magnetic stirrer and septum cap. A needle was then inserted into the septum so that H₂ was able to penetrate into the vial. The vial was inserted into an alloy plate and placed into a 300 ml steel Parr autoclave. The autoclave was purged three times with H₂ at 10 bar and finally pressurized to 10 bar. This was then placed into an aluminium block and kept at 25° C. for 16 hours. Finally, the samples were removed from the autoclave by addition of ethyl acetate to the crude mixture and subsequently filtered in order to remove the solid catalysts using a Celite pad. The product was isolated using a silica gel column chromatography (ethyl acetate/pentane=1/4).
Yield: 96%.
Optical purity: 99:1
   b) Reaction with 10 g of Pt/TiO₂

10 g of Pt/TiO₂ catalyst (6% by weight of Pt) and 250 ml of the substrate were added to a 450 ml stainless steel autoclave from Parr Instruments. The autoclave was purged three times with 5 bar of hydrogen. 40 bar of hydrogen was introduced into the autoclave and the reaction took place with mechanical stirring. The reaction temperature was 80° C. and the H₂ pressure was 40 bar. The reaction time is 24 h. After the gas consumption had ended, the reactor was cooled down to room temperature and the pressure was released. After the reaction, the solid catalyst and the product were removed by filtration.
Yield: >98%.

### c) Recycling of the Catalyst

The catalyst was washed with cyclohexane and then dried for 2 hours at 80° C. in the furnace. Subsequently, the catalyst was collected and thoroughly comminuted before being added to a vacuum system overnight in order to completely remove the adsorbed species. The recycled catalyst was used directly for the next run without further regeneration. Four cycles were carried out. More than 98% of the desired product was obtained in each pass.

As the experimental results show, the object is achieved by the process according to the invention.

1. Process comprising the process steps of:
a) initially charging a phthalate of formula (I): 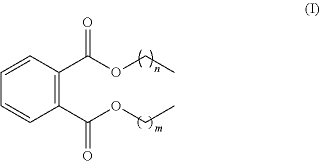
where n and m, each independently, are an integer from 0 to 12;
b) adding Pt/TiO₂;
c) feeding in H₂;
d) reacting the reaction mixture from a) to c), with conversion of the phthalate to a compound of formula (II):

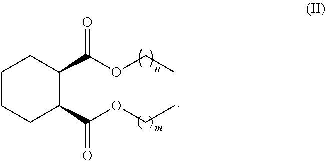

2. Process according to claim 1,
wherein n and m are the same number.

3. Process according to claim 1,
wherein n and m are a number from 4 to 8.

4. Process according to claim 1,
wherein the phthalate has the structure (1):

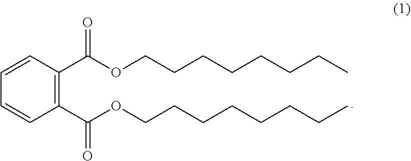 [SMILES: CCCCCCCCOC(=O)c1ccccc1C(=O)OCCCCCCCC]

5. Process according to claim 1,
wherein Pt/TiO₂ is added in a range from 0.1 mol % of Pt to 5 mol % of Pt (based on the phthalate).

6. Process according to claim 1,
wherein H₂ is fed in with a pressure in the range from 0.5 MPa (5 bar) to 10 MPa (100 bar).

7. Process according to claim 1,
wherein the reaction of the reaction mixture is effected at a temperature in the range from 20° C. to 120° C.

8. Process according to claim 1,
comprising the additional process step c′):
c′) adding a solvent.

9. Process according to claim 8,
wherein the solvent is cyclohexane.

10. Compound having the structure (1):

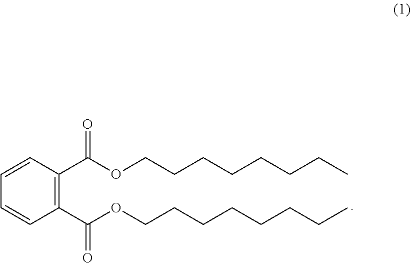 [SMILES: CCCCCCCCOC(=O)c1ccccc1C(=O)OCCCCCCCC]

11. Use of a Pt/TiO₂ catalyst for catalysis of a stereoselective hydrogenation of an aromatic compound.

12. Use according to claim 11,
wherein the aromatic compound is a phthalate.
